(19) United States
(12) Patent Application Publication
(10) Pub. No.: US 2022/0296205 A1
(43) Pub. Date: Sep. 22, 2022

(54) ULTRASOUND IMAGE-BASED DIAGNOSIS SYSTEM FOR CORONARY ARTERY LESION USING MACHINE LEARNING AND DIAGNOSIS METHOD OF SAME

(71) Applicants: THE ASAN FOUNDATION, Seoul (KR); UNIVERSITY OF ULSAN FOUNDATION FOR INDUSTRY COOPERATION, Ulsan (KR)

(72) Inventors: Soo Jin KANG, Seoul (KR); June Goo LEE, Seoul (KR); Ji Yuon KO, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 17/633,527

(22) PCT Filed: Aug. 5, 2020
(86) PCT No.: PCT/KR2020/010335

§ 371 Date: Feb. 7, 2022

(30) Foreign Application Priority Data
Aug. 5, 2019 (KR) 10-2019-0095167

(51) Int. Cl. A61B 8/06; A61B 8/12; G16H 50/20; G06T 7/00; G06T 7/11

(52) U.S. Cl. CPC A61B 8/06; A61B 8/12; G16H 50/20; G06T 7/0012; G06T 7/11; G06T 2207/30048; G06T 2207/30096; G06T 2207/10132; G06T 2207/20081; G06T 2207/30016; G06T 2207/30104

(57) ABSTRACT

Provided are a diagnostic system for predicting fractional flow reserve (FFR) through a machine learning algorithm based on an ultrasound image of a coronary artery and diagnosing the presence of a coronary artery lesion, and a diagnostic method thereof. The diagnostic method of diagnosing an ischemic lesion of a coronary artery includes: obtaining an intravascular ultrasound (IVUS) image of a coronary artery lesion of a patient; obtaining a mask image in which a vascular lumen is separated, by inputting the IVUS image into a first artificial intelligence model; extracting an IVUS feature from the mask image; and obtaining an FFR prediction value by inputting information including the IVUS feature into a second artificial intelligence model, and determining presence of an ischemic lesion.

### TECHNICAL FIELD

The present disclosure relates to an artificial intelligence (Al) system for simulating functions of the human brain, such as cognition, determination, etc., by using a machine learning algorithm, and to application thereof.

In detail, the present disclosure relates to a diagnostic system for predicting fractional flow reserve (FFR) through a machine learning algorithm based on an ultrasound image of a coronary artery and diagnosing the presence of a coronary artery lesion, and to a diagnostic method thereof.

### BACKGROUND ART

Recently, an artificial intelligence system that implements human-level intelligence has been used in various fields. Unlike existing rule-based smart systems, an artificial intelligence system is a system in which a machine learns, determines, and becomes smarter by itself. The more the artificial intelligence system is used, the better the recognition rate and the greater the accuracy in understanding user preferences, and thus, existing rule-based smart systems are gradually being replaced by deep learning-based artificial intelligence systems. Artificial intelligence technology includes machine learning (e.g., deep learning) and element technologies using machine learning.

On the other hand, intravascular ultrasound (IVUS) is a clinical test method for determining the morphological features of coronary artery lesions, observing arteriosclerosis, and achieving procedural stent optimization. However, conventional IVUS has a limitation in that it is impossible to determine whether or not a procedure is necessary because the presence of ischemia is not identified in stenotic lesions.

In particular, for ischemia evaluation of moderately stenotic lesions, fractional flow reserve (FFR) should be repeatedly performed during the procedure. In other words, although it is essential to check the presence of myocardial ischemia through the FFR to make a decision on the treatment for coronary artery stenotic lesions, an FFR test costs about 1 million won in Korean currency and takes time, and there is a risk of complications due to the administration of a drug called adenosine during the test.

In order to solve these problems, interest has been recently focused on the instantaneous wave-free ratio (iFR), which may diagnose the FFR with an accuracy of 80% without using adenosine, but the iFR also provides an insignificant cost reduction effect because expensive blood flow pressure lines need to be used. Also, recently, in the case of quantitative flow ratio (QFR) using cardiovascular angiography, it is known that the FFR is predicted with an accuracy of about 80% to about 85%, but QFR consumes a lot of time in that a result may only be obtained by three-dimensional (3D) restoration by matching two different images, and there are relatively many cases in which an appropriate image cannot be obtained.

Although guidelines recommend screening for ischemic lesions through an FFR test before the procedure, in reality, due to cost and time, in 70% or more of all surgical cases, decisions are made to perform a procedure based on only the form of stenosis on angiography or IVUS. Due to this, unnecessary stent procedures are being misused, and the need for a solution for this has emerged.

### DESCRIPTION OF EMBODIMENTS

### Technical Problem

The present disclosure is provided for the aforementioned need, and provides a system and method of predicting a fractional flow reserve (FFR) of less than 0.80 by using a machine learning model based on an intravascular ultrasound image and diagnosing ischemia without performing FFR during a procedure.

However, such a technical problem is merely an example, and the scope of the present disclosure is not limited thereto.

### Solution to Problem

According to an embodiment of the present disclosure, a diagnostic method of diagnosing an ischemic lesion of a coronary artery may include: obtaining an intravascular ultrasound (IVUS) image of a coronary artery lesion of a patient; obtaining a mask image, in which a vascular lumen is separated, by inputting the IVUS image into a first artificial intelligence model; extracting an IVUS feature from the mask image; and obtaining an FFR prediction value by inputting information including the IVUS feature into a second artificial intelligence model, and determining presence of an ischemic lesion.

Also, the mask image may be obtained by fusing pixels corresponding to an adventitia, a lumen, and a plaque of the coronary artery.

Also, the IVUS feature may include a first feature and a second feature, and the extracting of the IVUS feature may further include extracting the first feature based on the mask image, and calculating and obtaining the second feature based on the first feature.

Also, the information including the IVUS feature may include a clinical feature, and the determining of the presence of the ischemic lesion may further include obtaining the FFR prediction value by inputting the IVUS feature and the clinical feature into the second artificial intelligence model, and determining the presence of the ischemic lesion.

Also, the determining of the presence of the ischemic lesion may further include, when the FFR prediction value of a coronary artery lesion is less than or equal to 0.80, determining the coronary artery lesion as an ischemic lesion.

Moreover, according to an embodiment of the present disclosure, a recording medium may be a computer-readable recording medium having recorded thereon a program excutable by a processor to perform the deep-learning based diagnostic method of diagnosing the ischemic lesion.

Other aspects, features, and advantages of the disclosure will become better understood through the accompanying drawings, the claims and the detailed description.

### Advantageous Effects of Disclosure

According to an embodiment of the present disclosure as described above, a system of the present disclosure may predict ischemia with a high accuracy of 81%.

Also, according to the present disclosure, a hemodynamic ischemia state may be diagnosed only by intravascular ultrasound (IVUS) without using a fractional flow reserve (FFR) pressure wire, thereby reducing time and cost.

In addition, according to the present disclosure, FFR may be quickly and accurately predicted by using artificial intelligence, and it may be determined whether treatment is necessary through ischemia diagnosis during a procedure, thereby reducing indiscriminate stenting.

The scope of the present disclosure is not limited by these effects.

### BRIEF DESCRIPTION OF DRAWINGS

FIG. 1 is a system diagram illustrating an ischemic lesion diagnostic system according to an embodiment of the present disclosure.

FIG. 2 is a simple block diagram illustrating components of an ischemic lesion diagnostic device according to an embodiment of the present disclosure.

FIG. 3 is a simple flowchart illustrating an ischemic lesion diagnostic method according to an embodiment of the present disclosure.

FIG. 4 is a diagram for describing a set for training an artificial intelligence model and baseline characteristics, according to an embodiment of the present disclosure.

FIGS. 5A and 5B are diagrams for describing obtaining a vascular lumen separation image according to an embodiment of the present disclosure.

FIGS. 6A and 6B are diagrams for describing an intravascular ultrasound (IVUS) feature according to an embodiment of the present disclosure.

FIG. 7 is a diagram for describing the top 20 important characteristics for each algorithm, according to an embodiment of the present disclosure.

FIGS. 8A and 8B illustrate the results of performing 5-fold cross-validation on a training set and a test set, respectively, according to an embodiment of the present disclosure.

FIG. 9 illustrates the performance and 95% confidence interval of 200 bootstrap replicas.

FIG. 10 is a diagram illustrating the results of ROC analysis using various machine learning (ML) models.

FIG. 11 is a diagram illustrating the misclassification frequency of each algorithm according to a range of fractional flow reserve (FFR) values.

FIG. 12 is a block diagram illustrating a trainer and a recognizer, according to various embodiments of the present disclosure.

### MODE OF DISCLOSURE

Hereinafter, various embodiments of the present disclosure will be described with reference to the accompanying drawings. As the present disclosure allows for various changes and numerous embodiments, certain embodiments will be illustrated in the drawings and described in the detailed description. However, various embodiments are not intended to limit the present disclosure to certain embodiments, and should be construed as including all changes, equivalents, and/or alternatives included in the spirit and scope of various embodiments of the present disclosure. With regard to the description of the drawings, similar reference numerals may be used to refer to similar components.

Expressions such as “include” or “may include” that may be used in various embodiments of the present disclosure specify the presence of a corresponding function, operation, or component, and do not preclude the presence or addition of one or more functions, operations, or components. Also, it will be understood that terms such as “include” or “comprise” as used in various embodiments of the present disclosure specify the presence of stated features, numbers, steps, operations, components, parts, and combinations thereof, but do not preclude in advance the presence or addition of one or more other features, numbers, steps, operations, components, parts, combinations thereof.

A term such as “or” as used in various embodiments of the present disclosure may include any and all possible combinations of words listed together. For example, an expression such as “A or B” may include “A,” “B,” or both “A” and “B.”

Expressions such as “first,” “second,” “primarily,” or “secondarily” as used in various embodiments of the present disclosure may represent various components and do not limit corresponding components. For example, the aforementioned expressions do not limit the order and/or importance of the corresponding components. The aforementioned expressions may be used to distinguish one component from another. For example, both a first user device and a second user device refer to user devices and represent different user devices. For example, without departing from the scope of various embodiments of the present disclosure, a first component may be referred to as a second component, and similarly, a second component may be referred to as a first component.

It will be understood that when a component is referred to as being “connected” or “coupled” to another component, it may be directly connected or coupled to the other component, or intervening components may exist between the component and the other component. On the other hand, it will be understood that when a component is referred as being “directly connected” or “directly coupled” to another component, intervening components may not exist between the component and the other component.

Terms such as “module,” “unit,” and “part” as used in the embodiments of the present disclosure refer to components that perform at least one function or operation, and the components may be implemented as hardware or software or as a combination of hardware and software. Also, a plurality of “modules,” “units,” and “parts” may be integrated into at least one module or chip and implemented as at least one processor, except when each of the modules, units, and parts needs to be implemented as individual specific hardware.

Terms used in various embodiments of the present disclosure are merely used to describe certain embodiments, and are not intended to limit various embodiments of the present disclosure. As used herein, the singular forms “a,” “an,” and “the” are intended to include the plural forms as well, unless the context clearly indicates otherwise.

Unless otherwise defined, all terms used herein including technical or scientific terms have the same meanings as commonly understood by those of ordinary skill in the art to which various embodiments of the present disclosure pertain.

Terms such as those defined in commonly used dictionaries should be interpreted as having meanings consistent with the meanings in the context of the related art, and should not be interpreted in an idealized or overly formal sense, unless explicitly defined in various embodiments of the present disclosure.

Hereinafter, various embodiments of the present disclosure will be described in detail with reference to the accompanying drawings.

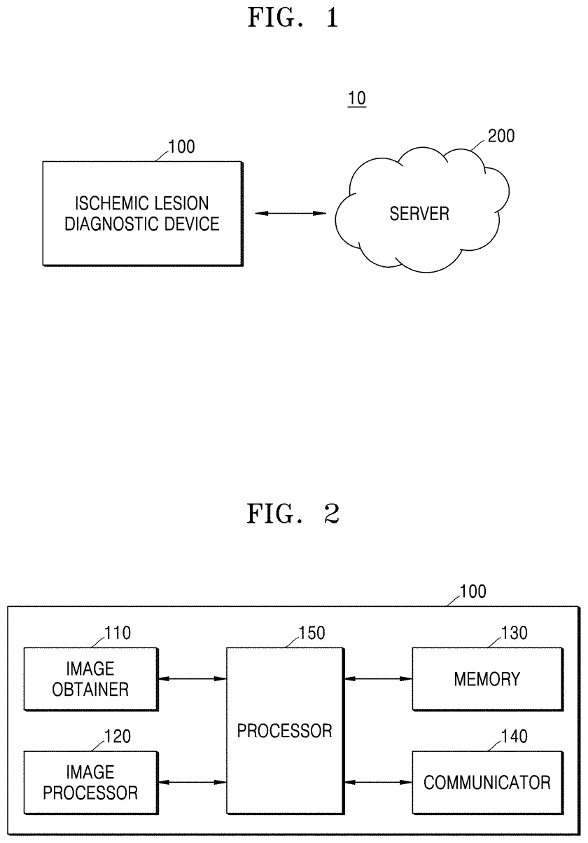

FIG. 1 is a system diagram illustrating an ischemic lesion diagnostic system according to an embodiment of the present disclosure.

Referring to FIG. 1, an ischemic lesion diagnostic system 10 of the present disclosure may include an ischemic lesion diagnostic device 100 and a server 200.

The ischemic lesion diagnostic device 100 is a device for predicting and diagnosing an ischemic lesion occurring in a patient's coronary artery.

The presence of an ischemic lesion may not be determined based on the presence of a stenotic coronary artery in appearance, but may be determined based on the presence of functional stenosis. That is, even though there is stenosis in appearance, the stenosis may not be determined as an ischemic lesion. Fractional flow reserve (FFR) is defined as a ratio of the maximum coronary flow in an artery with stenosis to the maximum coronary flow in the same artery without stenosis. Therefore, it may be determined through FFR whether the ischemic lesion is caused by functional stenosis.

Accordingly, the ischemic lesion diagnostic device 100 may diagnose the presence of an ischemic lesion by predicting a value of FFR of a coronary artery. In detail, when the FFR is 0.80, it indicates that the stenotic coronary artery is supplying 80% of its normal maximum flow, and the ischemic lesion diagnostic device 100 may determine that the ischemic lesion has functional stenosis in the coronary artery when the FFR is less than or equal to 0.80.

The server 200 is at least one external server for training and refining an artificial intelligence (Al) model and performing prediction by using an artificial intelligence model.

The server 200 according to an embodiment of the present disclosure may include a first Al model for extracting a vascular boundary image for an intravascular ultrasound (IVUS) image and a second Al model for predicting FFR of a blood vessel.

In this case, the first Al model may be a model that outputs a vascular lumen separation image or a mask image when the IVUS image is input. Also, when various pieces of feature information about blood vessels and a patient are input, the second Al model may determine the presence of an ischemic lesion if an FFR value of a coronary artery lesion is less than or equal to 0.80. In this case, the pieces of feature information may include, but are not limited to, a morphological feature, a computational feature, and a clinical feature on the IVUS image. More details on this will be described below.

Though FIG. 1 illustrates that the ischemic lesion diagnostic device 100 and the server 200 are implemented as separate components, the ischemic lesion diagnostic device 100 and the server 200 may be implemented as a single component according to an embodiment of the present disclosure. That is, according to an embodiment, the ischemic lesion diagnostic device 100 may be an on-device Al device that directly trains and refines the first Al model and the second Al model.

FIG. 2 is a simple block diagram illustrating components of the ischemic lesion diagnostic device 100 according to an embodiment of the present disclosure.

Referring to FIG. 2, the ischemic lesion diagnostic device 100 may include an image obtainer 110, an image processor 120, a memory 130, a communicator 140, and a processor 150 electrically connected to and configured to control the aforementioned components.

The image obtainer 110 may obtain IVUS image data through various resources. For example, the image obtainer 110 may be implemented as a commercial scanner and may obtain an IVUS image by scanning the inside of a coronary artery. Image data obtained by the image obtainer 110 may be processed by the image processor 120.

The image processor 120 may process the image data obtained by the image obtainer 110. The image processor 120 may perform various image processes, such as decoding, scaling, noise reduction, frame rate conversion, resolution change, and the like, on the image data.

The memory 130 may store various data for an overall operation of the ischemic lesion diagnostic device 100, such as a program for processing or control by the processor 150, or the like. The memory 130 may store a plurality of application programs (or applications) driven by the ischemic lesion diagnostic device 100, data and instructions for operations of the ischemic lesion diagnostic device 100, etc. At least some of the application programs may be downloaded from an external server through wireless communication.

Also, some of the application programs may exist on the ischemic lesion diagnostic device 100 from the time of shipment for basic functions of the ischemic lesion diagnostic device 100. The application programs may be stored in the memory 130 and driven by the processor 150 to perform operations (of functions) of the ischemic lesion diagnostic device 100. In particular, the memory 130 may be implemented as, for example, an internal memory such as a read-only memory (ROM), a random access memory (RAM), etc. included in the processor 150, or may be implemented as a memory separate from the processor 150.

The communicator 140 may be a component that communicates with various types of external devices according to various types of communication methods. The communicator 140 may include at least one of a Wi-Fi chip, a Bluetooth chip, a wireless communication chip, and a near field communication (NFC) chip. The processor 150 may communicate with the server 200 or various external devices using the communicator 140.

In particular, in the case of using a Wi-Fi chip or a Bluetooth chip, various types of connection information such as a service set identifier (SSID) and a session key are first transmitted and received, and then, after a communication connection is established by using the same, various types of information may be transmitted and received. The wireless communication chip refers to a chip that performs communication according to various communication standards such as IEEE, Zigbee, 3rd generation (3G), 3rd generation partnership project (3GPP), and long term evolution (LTE). The NFC chip refers to a chip operating in an NFC method using a 13.56 MHz band among various RF-ID frequency bands such as 135 kHz, 13.56 MHz, 433 MHz, 860 MHz to 960 MHz, 2.45 GHz, and the like.

The processor 150 is configured to generally control the ischemic lesion diagnostic device 100. In detail, the processor 150 controls an overall operation of the ischemic lesion diagnostic device 100 by using various programs stored in the memory 130 of the ischemic lesion diagnostic device 100. For example, the processor 150 may include a central processing unit (CPU), a RAM, a ROM, and a system bus. In this case, the ROM is a component in which an instruction set for system booting is stored, and the CPU copies an operating system (O/S) stored in a memory of a remote control device 100 to the RAM according to an instruction stored in the ROM, and executes the O/S to boot the system. When booting is completed, the CPU may perform various operations by copying and executing various applications stored in the memory 130. Although it has been described above that the processor 150 includes only one CPU, the processor 150 may be implemented as a plurality of CPUs (or digital signal processors (DSPs), systems on chip (SoCs), etc.) upon implementation.

According to an embodiment of the present disclosure, the processor 150 may be implemented as a DSP, a microprocessor, or a time controller (TCON), which processes a digital signal. However, the processor 150 is not limited thereto and may include one or more of a CPU, a micro controller unit (MCU), a micro processing unit (MPU), a controller, an application processor (AP), a communication processor (CP), or an advanced reduced instruction set computer (RISC) machine (ARM) processor, and may be defined by a corresponding term. Also, the processor 150 may be implemented as a SoC or large scale integration (LSI) having a built-in processing algorithm, or may be implemented in the form of a field programmable gate array (FPGA).

The processor 150 may include a feature extractor (not shown) and an ischemic lesion determiner (not shown).

The feature extractor may obtain a mask image, in which a vascular lumen is separated, by inputting an IVUS image of a patient's coronary artery lesion, which is obtained by an image obtainer, into the first Al model, and extract an IVUS feature from the mask image. The ischemic lesion determiner may obtain an FFR prediction value by inputting information including the IVUS feature into the second Al model, and determine the presence of an ischemic lesion.

The feature extractor (not shown) and the ischemic lesion determiner (not shown) according to an embodiment of the present disclosure may be implemented through a separate software module stored in the memory 130 and driven by the processor 150. Each software module may perform one or more functions and operations described herein. Also, each component may be implemented as a separate module, or components may be implemented as a single module.

Moreover, as described above, according to another embodiment of the present disclosure, the feature extractor (not shown) and the ischemic lesion determiner (not shown) may be components included in a processor (not shown) in the server 200.

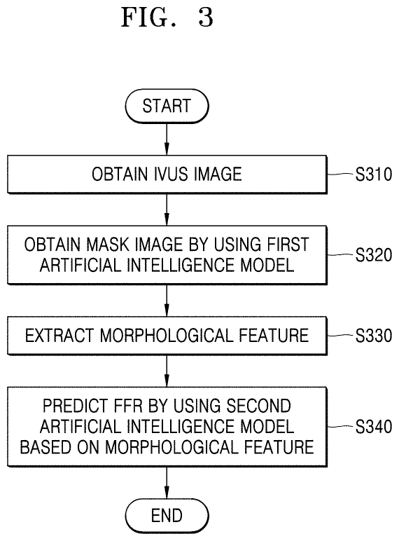

FIG. 3 is a simple flowchart illustrating an ischemic lesion diagnostic method according to an embodiment of the present disclosure.

The ischemic lesion diagnostic system 10 of the present disclosure may obtain an IVUS image (S310). In this case, the IVUS image may be an image including a plurality of frames (e.g., 2,000 frames to 4,000 frames) according to the length of an ischemic lesion from a patient with coronary artery disease.

The IVUS image may be obtained by administrating 0.2 mg of nitroglycerin into a coronary artery, and then performing a grayscale IVUS imaging by using a commercial scanner configured with a motorized transducer pullback (0.5 mm/s) and a 40 MHz transducer rotating within a 3.2 F imaging enclosure.

The ischemic lesion diagnostic system 10 may obtain a mask image in which a vascular lumen boundary is separated, by using a first Al model (S320).

In this case, the first Al model may be a machine learning model trained to output a vascular lumen separation image when the IVUS image is input. In this regard, the first Al model may be trained by using, as training data, a vascular lumen separation image whose outline is manually set at intervals of 0.2 mm of a blood vessel (about every 12 frames).

In detail, vascular lumen separation may be performed by using an interface between the lumen and the anterior edge of the intima. The vascular lumen separation may be performed based on that a separated interface at a boundary between the intima-media and the adventitia substantially matches the position of an external elastic membrane (EEM).

The ischemic lesion diagnostic system 10 may extract various IVUS features from the mask image in which a vascular boundary is automatically separated (S330). The ischemic lesion diagnostic system 10 may obtain an FFR prediction value through a second Al model based on information including the IVUS features, and determine the presence of an ischemic lesion (S340). In this case, the IVUS features may include an IVUS morphological feature and an IVUS computational feature.

In detail, the ischemic lesion diagnostic system 10 may extract an IVUS morphological feature or a first feature based on the mask image and calculate and obtain an IVUS computational feature or a second feature based on the IVUS morphological feature.

Moreover, the information including the IVUS features may include a clinical feature, and an FFR prediction value may be obtained by inputting the IVUS features and the clinical feature into the second Al model, and the presence of an ischemic lesion may be determined. In particular, when the FFR prediction value of a coronary artery lesion is less than or equal to 0.80, the ischemic lesion diagnostic system 10 may determine the lesion as an ischemic lesion.

In this case, the clinical feature may include age, gender, body surface area, lesion segment (hereinafter, referred to as involved segment), involvement of proximal left anterior descending artery (LAD), and vessel type.

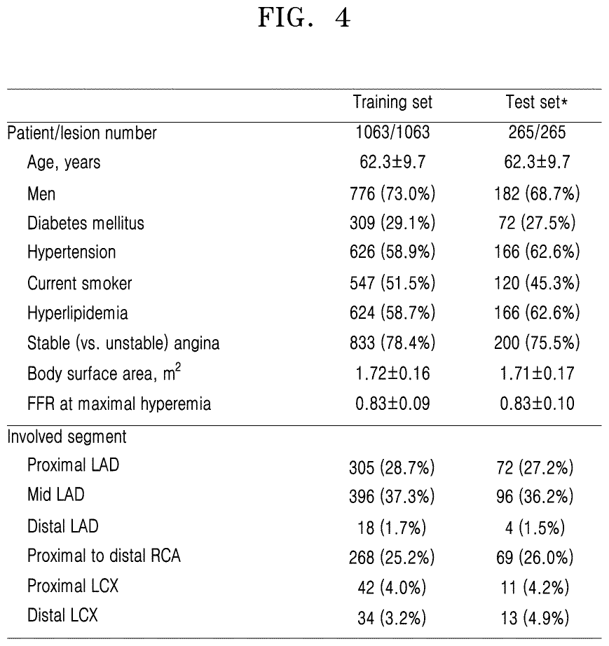

FIG. 4 is a diagram for describing a training set for training an Al model, a test set, and baseline characteristics, according to an embodiment of the present disclosure.

From November 2009 to July 2015, 1657 patients who underwent invasive coronary angiography were evaluated. In this case, patients may be those evaluated through IVUS and FFR prior to procedures as patients with a moderate lesion visually defined by angiographic DS of about 40% to about 80%. When IVUS and FFR were measured for multiple lesions, patients with primary coronary lesions with the lowest FFR value were selected.

Among the patients, except for a total of 329 patients, including 77 patients with a tandem lesion, 95 patients with a stent in a target blood vessel, 4 patients who are side-branch evaluated, 49 patients with left main coronary artery stenosis (angiographic DS>30%), 59 patients with incomplete IVUS, 12 patients with chronic obstructive pulmonary disease, 8 patients with severe myocardial and regional wall movement abnormality at a wound site, and 9 patients with a technical error in a video file, 1328 patients with non-left main coronary artery stenosis were selected for the cohort of the present retrospective analysis.

The aforementioned patients were assigned to the training set and the test set, respectively, in a ratio of 4:1. That is, information about 1063 random patients was used to train an Al model, and information about 265 random patients, who do not overlap the 1063 random patients, was used to evaluate the performance of the Al model.

The baseline characteristics may include a patient characteristic and an involved segment characteristic. In this case, the patient characteristic may include age, gender, smoking state, body surface area, FFR at maximal hyperemia (FFR), and the like. The involved segment characteristic may be a region of a coronary artery in which a stenotic lesion has occurred, and may include a left anterior descending artery (LAD), a left circumflex artery (LCX), and a right coronary artery (RCA). Referring to FIG. 4, 67.1 (891 patients) of the involved segment were LAD, 7.5% (100 patients) thereof were LCX, and 25.4% (337 patients) thereof were RCA. The ischemic lesion diagnostic system 10 of the present disclosure may train and test the Al model by preventing a sample difference between the training set and the test set from being large.

Moreover, in the training set, an FFR of less than 0.80 was more frequently shown in men than in women (38.8% vs. 24.0%, p<0.001). Also, an FFR of less than or equal to 0.80 were more frequent at younger age (60.2±9.8 vs. 63.4±9.4 years old, p<0.001) and greater body surface area (1.76±0.16 vs. 1.71±0.16 m2, p<0.001).

In the involved segment, 39.5% of the proximal LAD had an FFR of less than or equal to 0.80, and 22.9% thereof had an FFR of greater than 0.80 (p<0.001). Also, 44.4% of LAD had an FFR of less than 0.80, and 14.6% of RCA and 15.8% of LCX had an FFR of less than 0.80.

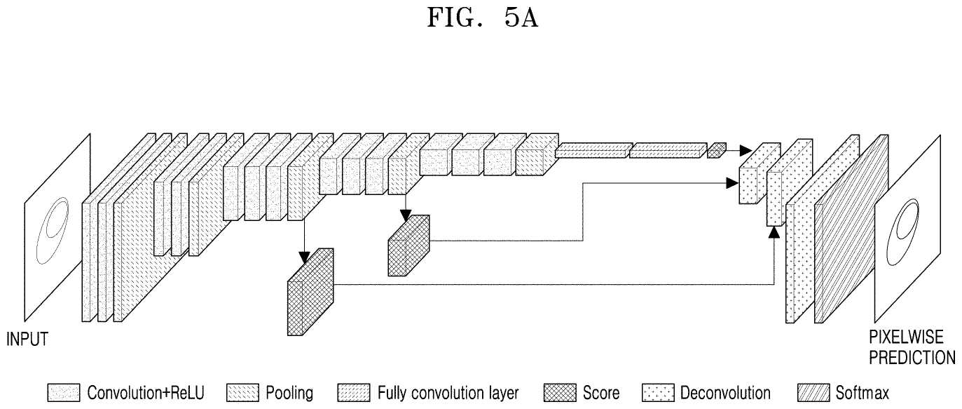

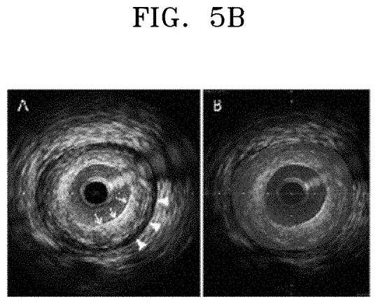

FIGS. 5A and 5B are diagrams for describing obtaining a vascular lumen separation image according to an embodiment of the present disclosure.

FIG. 5A is a diagram illustrating a first Al model according to an embodiment of the present disclosure.

The first Al model may decompose a frame included in an IVUS image by using a fully convolutional network (FCN) previously trained from an ImageNet database. Then, the first Al model applies skip connections to an FCN-VGG16 model that combines hierarchical characteristics of convolutional layers of different scales. By combining three predictions of 8, 16, and 32 pixel strides through the skip connections, the first Al model may output an output with improved spatial precision through the FCN-VGG16 model.

Moreover, the first Al model may be converted into an RGB color format by resampling the IVUS image to a size of 256×256 as a pre-processing operation. A central image and a neighboring image having a displacement value different from that of the central image may be merged into a single RGB image, and 0, 1, and 2 frames are used as three displacement values.

In detail, a cross-sectional image may be divided into 3 segments, (i) an adventitia (coded as “0”) including pixels outside an EEM, (ii) a lumen (coded as “1”) including pixels within a lumen boundary, and (iii) a plaque (coded as “2”) including pixels between the lumen boundary and the EEM. In order to correct pixel dimensions, grid lines may be automatically obtained from the IVUS image, and cell intervals may be calculated.

The first Al model or an FCN-all-at-once-VGG16 model may be trained for each displacement setting by using preprocessed image pairs (e.g., a 24-bit RGB color image and an 8-bit gray mask). As described above, the first Al model may output one mask image by fusing three extracted masks.

FIG. 5B illustrates a vascular lumen separation image according to an embodiment of the present disclosure.

Referring to FIG. 5B, when an IVUS image (A) of a coronary artery is input into the first Al model of the present disclosure, a vascular lumen separation image (B) or a mask image in which the lumen of the coronary artery and the adventitia are separated may be obtained.

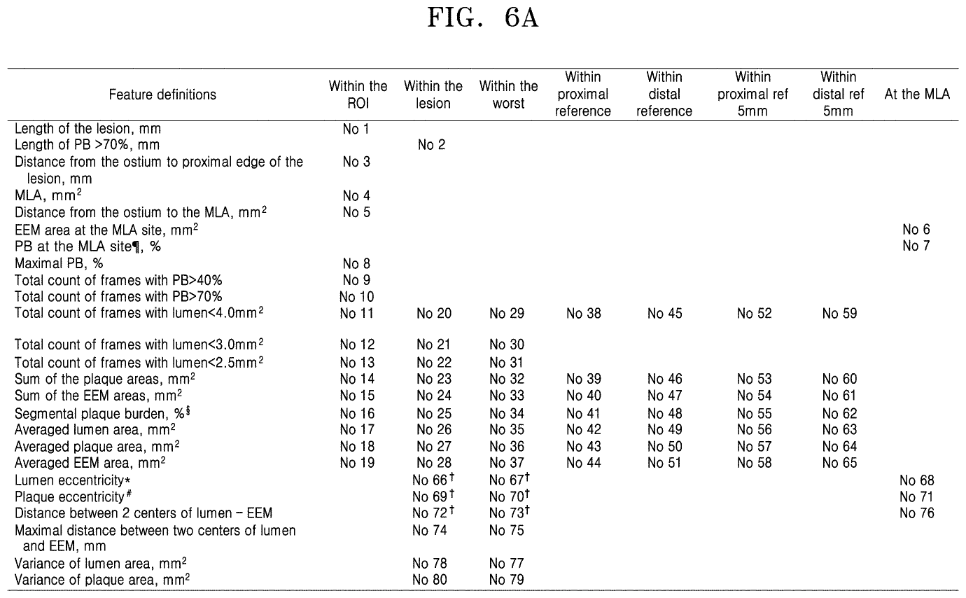

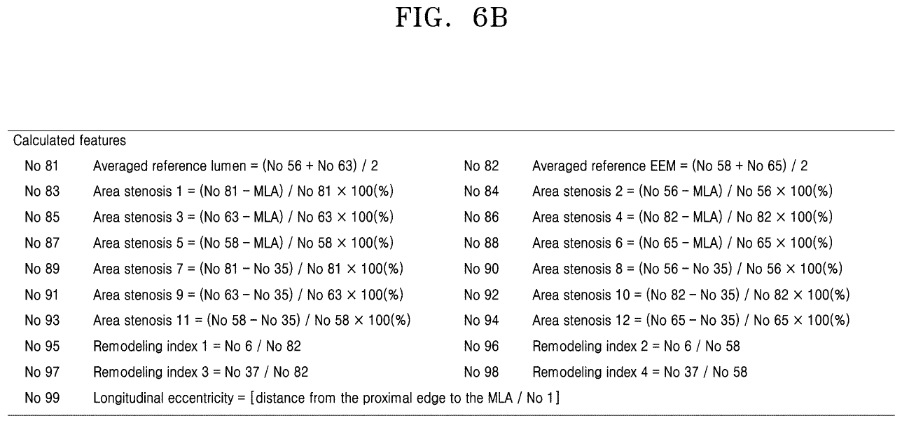

FIGS. 6A and 6B are diagrams for describing an IVUS feature according to an embodiment of the present disclosure.

Referring to FIGS. 6A and 6B, the IVUS feature for training an Al model of the present disclosure may be identified. The IVUS feature may be a morphological feature that may be identified through an IVUS image (or a mask image) and a feature calculated from the morphological feature.

FIG. 6A illustrates an example of 80 morphological features (hereinafter, referred to as angiographic features) or first features of a blood vessel that may be extracted through an IVUS image. For example, referring to FIG. 6A, the ischemic lesion diagnostic system of the present disclosure may extract angiographic features such as a lesion length feature in a region of interest (ROI) (No. 1), a plaque burden (PB) length feature in a lesion (No. 2), etc.

FIG. 6B illustrates an example of 19 calculated features or second features for a blood vessel. For example, referring to FIG. 6B, an averaged reference lumen feature (No. 81) may be obtained by calculating an average of an angiographic feature (No. 56) for an average lumen based on proximal 5 mm and an angiographic feature (No. 63) for an average lumen based on distal 5 mm. That is, the averaged reference lumen feature (No. 81) may be calculated by Equation 1 below.

Averaged reference lumen(No.81)=(No.56+No.63)/2 [Equation 1]

As another example, a stenosis area 1 feature (No. 83) may be calculated by using the averaged reference lumen feature (No. 81) and a minimal lumen area (MLA). That is, the stenosis area 1 feature (No. 83) may be calculated by Equation 2 below.

Area stenosis1(No.82)=(No.81−MLA)/(No.81)×100% [Equation 2]

The MLA may be defined by selecting a frame that exhibits the smallest lumen area and PB of greater than 40%. A lesion including an MLA site may be defined as a segment with a PB of less than 40% and a segment of PB of greater than 40% with fewer than 25 consecutive frames (<5 mm). The PB may be calculated as a percentage (%) value of (EEM area−lumen area) divided by the EEM area.

The ROI may be defined as a segment from the ostium to a segment 10 mm away from the lesion. A proximal reference may be defined as a segment between the start of the ROI and a proximal edge of the lesion, and a distal reference may be defined as a segment between a distal edge of the lesion and the end of the ROI. The expression “based on proximal or distal 5 mm” may refer to within a proximal or distal 5 mm portion of the lesion. The worst segment may be defined as a 4 mm portion that is 2 mm proximal and 2 mm distal from the MLA site.

Moreover, the ischemic lesion diagnostic system 10 according to an embodiment of the present disclosure may use a total of 105 features including the aforementioned 99 IVUS features (80 angiographic features and 19 computational features) and 6 clinical features, as training data for machine learning of the second Al model. In this case, the clinical features may include age, gender, body surface area, involved segment, involvement of proximal LAD, and vessel type.

Also, the ischemic lesion diagnostic system 10 according to an embodiment of the present disclosure may train the second Al model by using, as training data, the 105 features for the IVUS image and FFR values of patients (e.g., the training set of FIG. 4) corresponding to the IVUS image. The second Al model trained through this may output prediction values of the FFR values when the 105 features for the IVUS image are input.

With regard to obtaining of a patient's FFR, “equalizing” was performed with a guide wire sensor positioned at the tip of a guide catheter, and a 0.014-inch FFR pressure guide ware was advanced to the periphery of a stenosis site. The FFR was measured at a maximal hyperemia state induced by intravenous infusion of adenosine. That is, in order to hemodynamically improve detection of stenosis, the infusion was increased from 140 μg/kg/min to 200 μg/kg/min through a central vein. After hypertensive compression recording is performed, FFR may be obtained as a ratio of distal coronary arterial pressure to normal perfusion pressure (aortic pressure) at the maximal hyperemia state.

Moreover, the second Al model of the present disclosure may be implemented through a plurality of algorithms. For example, the second Al model may be implemented through an ensemble of six Al algorithms, but is not limited thereto.

The six Al algorithms of the second Al model according to an embodiment of the present disclosure may be evaluated as the performance of a binary classifier for separating FFRs of less than equal to 0.80 and FFRs of greater than 0.80. In this case, the six Al algorithms may include L2 penalized logistic regression, artificial neural network (ANN), random forest, AdaBoost, CatBoost, and support vector machine (SVM), but are not limited thereto. Also, the aforementioned six Al algorithms may be independently trained with at least 200 training-test random splits generated by using a bootstrap method. The importance of each feature for FFR prediction of each algorithm may be different.

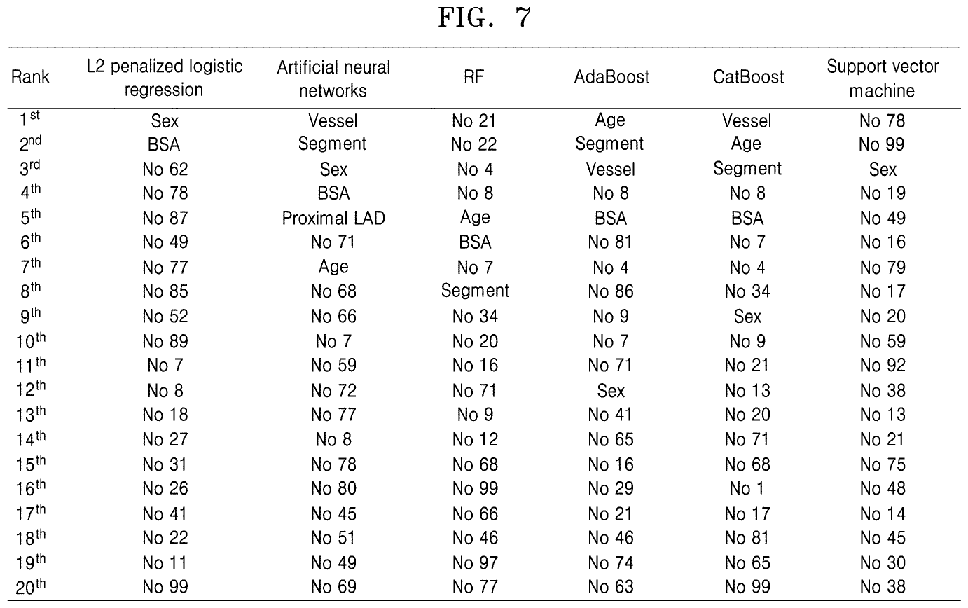

FIG. 7 is a diagram for describing the top 20 important characteristics for each algorithm, according to an embodiment of the present disclosure.

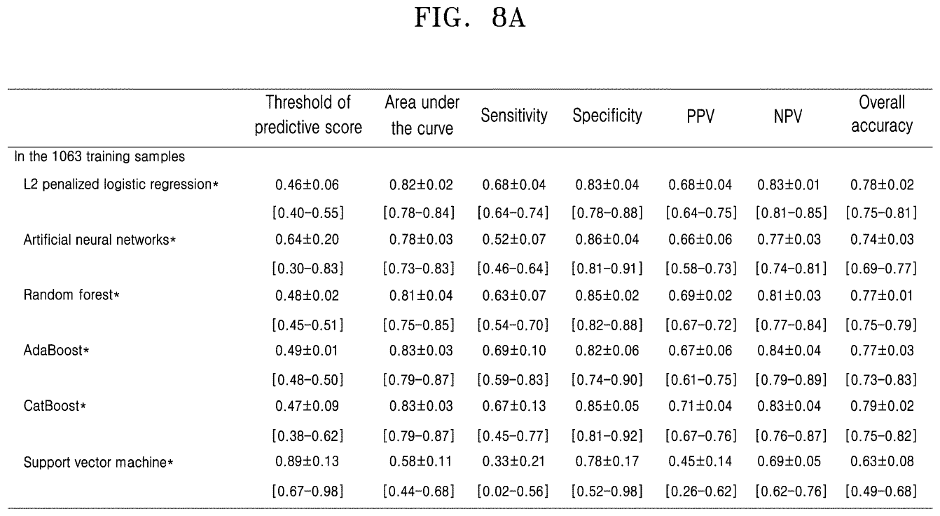

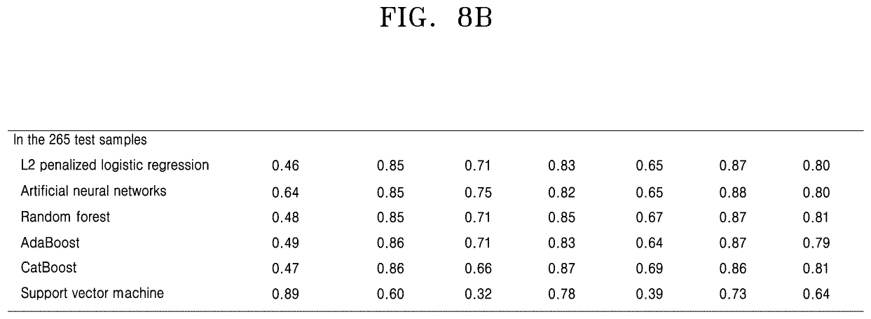

Referring to FIG. 7, the 20 most important features for predicting a lesion with an FFR of less than 0.80 are shown for each algorithm. For such classification, 5-fold cross-validation of all clinical features and IVUS features of training data as shown in FIGS. 8A and 8B may be used.

The 5-fold cross-validation means that a training set is divided into 5 partitions so that partitions do not overlap each other, and when one partition becomes a test set, remaining 4 partitions become a training set and are used as training data. In this case, the test may be repeated 5 times so that each of the 5 partitions becomes a test set once. Accuracy is calculated as an average of accuracies over 5 tests. In order to reduce variability, multiple cross-validations may be performed times and may be averaged.

FIGS. 8A and 8B illustrate the results of performing 5-fold cross-validation on a training set and a test set, respectively, according to an embodiment of the present disclosure.

Referring to FIGS. 8A and 8B, it may be seen that the diagnostic accuracy of predicting an FFR of less than 0.80 in L2 penalized logistic regression, ANN, random forest, and CatBoost algorithms exceeds 80% (AUC: 0.85 to 0.86).

A receiver operating characteristic curve (ROC) considering an entire range of possible probability values (from 0 to 1) shows a value of 0.5 when there is no predictive power and a value of 1 when complete prediction and classification are performed.

Moreover, the ischemic lesion diagnostic system of the present disclosure may perform 5-fold cross-validation several times. The accuracy of 5-fold cross-validation may then be calculated by averaging the accuracies of the tests.

As described above, for non-biased performance evaluation, a classifier constructed through the training set applies a non-overlapping test set. In particular, through bootstrapping, each algorithm of the present disclosure may be independently trained on 200 training-test random data splits in a 4:1 ratio. An average performance and a 95% confidence interval of 200 bootstrap replicas may be expressed as mean±standard deviation for each training-test set.

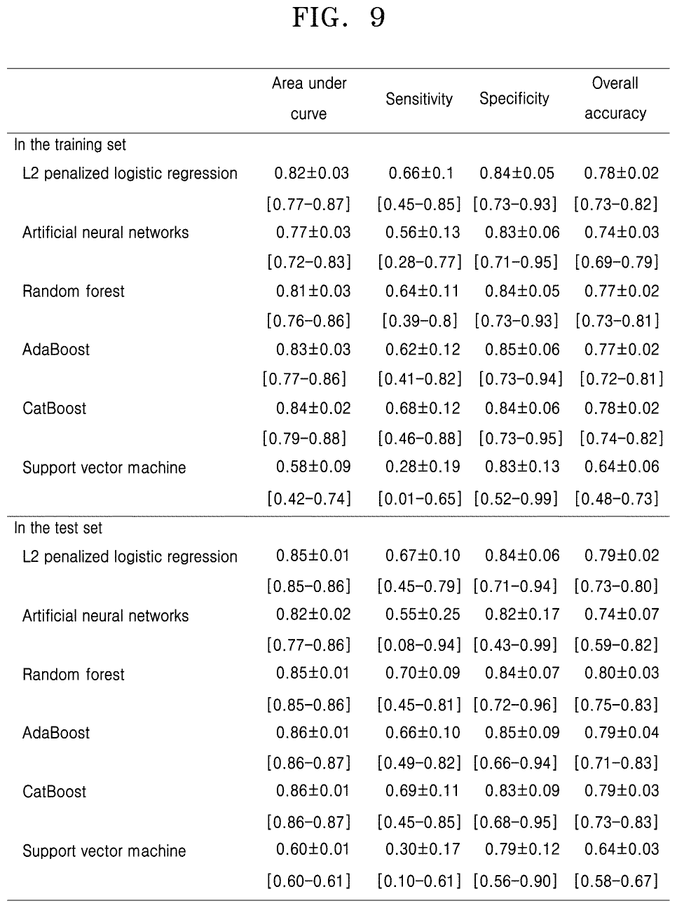

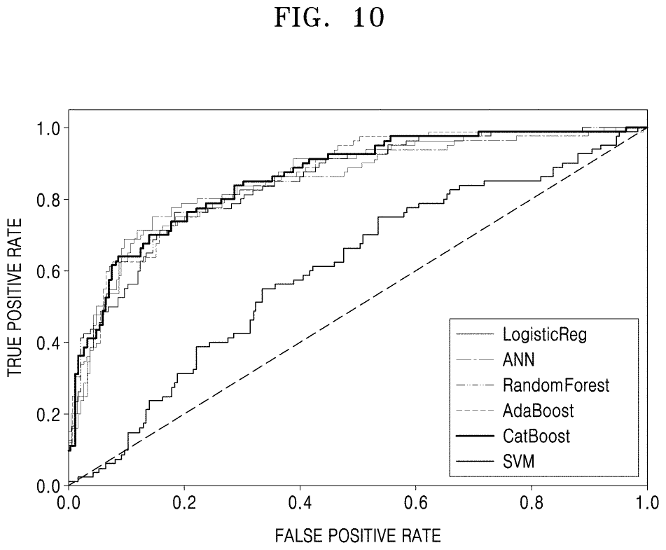

FIG. 9 illustrates the performance and 95% confidence interval of 200 bootstrap replicas, and FIG. 10 is a diagram illustrating the results of ROC analysis using various machine learning (ML) models.

Referring to FIGS. 9 and 10, all other algorithms except for an SVM algorithm may identify an overall accuracy of 70% or more in a 95% confidence interval.

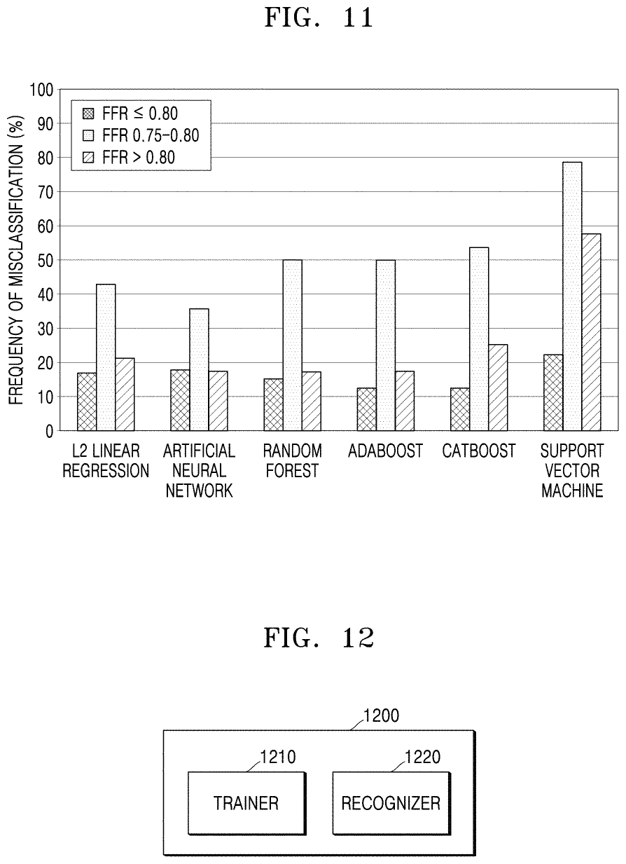

FIG. 11 is a diagram illustrating the misclassification frequency of each algorithm according to a range of FFR values.

When 28 lesions with local FFR values (0.75 to 0.80) were excluded, the overall accuracy of the test set was found to be 86.5% for AdaBoost, 82.3% for ANN, 84.3% for random forest, 82.3% for L2 penalized logistic regression, and 70% for SVM.

In summary, when lesions were classified by patients with an FFR of less than or equal to 0.80 and an FFR of greater than 0.80, the overall accuracy of the other algorithms except for the SVM algorithm was found to be about 80%.

That is, by using L2 penalized logistic regression, random forest, AdaBoost, and CatBoost algorithms, an average accuracy of 200 bootstrap replicates is about 79% to about 80%, with an average area under curve (AUC) of about 0.85 to about 0.86. In this case, when an FFR value was between 0.75 and 0.80, the frequency of misclassification was high. Excluding 28 lesions with an FFR of 0.75 to 0.80, the accuracy was found to be 87% for AdaBoost, 85% for CatBoost, 82% for ANN, 84% for random forest, and 82% for L2 penalized logistic regression.

FIG. 12 is a block diagram illustrating a trainer and a recognizer, according to various embodiments of the present disclosure.

Referring to FIG. 12, a processor 1200 may include at least one of a trainer 1210 and a recognizer 1220. The processor 1200 of FIG. 12 may correspond to the processor 150 of the ischemic lesion diagnostic device 100 of FIG. 2 or the processor (not shown) of the server 200.

The trainer 1210 may generate or train a recognition model having a criterion for determining a certain situation. The trainer 1210 may generate a recognition model having a determination criterion by using collected training data.

As an example, the trainer 1210 may generate, train, or refine an object recognition model having a criterion for determining what kind of lumen of a blood vessel included in an IVUS image is, by using various IVUS images as training data.

As another example, the trainer 1210 may generate, train, or refine a model having a criterion for determining an FFR value for an input feature by using various IVUS features, clinical features, and FFR value information as training data.

The recognizer 1220 may estimate target data by using certain data as input data of the trained recognition model.

As an example, the recognizer 1220 may obtain (estimate, or infer) a mask image in which a vascular lumen included in an image is separated, by using various IVUS images as input data of the trained recognition model.

As another example, the recognizer 1220 may estimate (determine, or infer) an FFR value by applying various IVUS features and clinical features to the trained recognition model. In this case, the FFR value may be obtained as a plurality of FFR values according to priority.

At least a portion of the trainer 1210 and at least a portion of the recognizer 1220 may be implemented as a software module or manufactured in the form of at least one hardware chip and mounted in an electronic device. For example, at least one of the trainer 1210 and the recognizer 1220 may be manufactured in the form of a dedicated hardware chip for AI, or may be manufactured as a part of an existing general-purpose processor (e.g., a CPU or an AP) or a graphics-only processor (e.g., a graphics processing unit (GPU)) and mounted on various electronic devices or object recognition devices described above. In this case, the dedicated hardware chip for Al is a dedicated processor specialized in probability calculation, and has higher parallel processing performance than the existing general-purpose processor, and thus may quickly process calculation tasks in Al fields, such as machine learning.

When each of the trainer 1210 and the recognizer 1220 are implemented as a software module (or a program module including instructions), the software module may be stored in a computer-readable non-transitory computer-readable medium. In this case, the software module may be provided by an OS or may be provided by a certain application. Alternatively, a part of the software module may be provided by an OS, and other parts thereof may be provided by a certain application.

In this case, the trainer 1210 and the recognizer 1220 may be mounted on one electronic device or may be mounted on separate electronic devices, respectively. For example, one of the trainer 1210 and the recognizer 1220 may be included in the ischemic lesion diagnostic device 100, and the other thereof may be included in the server 200. Also, the trainer 1210 and the recognizer 1220 may be configured so that model information constructed by the trainer 1210 may be provided to the recognizer 1220 and data input into the recognizer 1220 may be provided to the trainer 1210 as additional training data through wired or wireless communication.

Moreover, the aforementioned methods according to various embodiments of the present disclosure may be implemented in the form of an application that may be installed in an existing electronic device.

Moreover, according to an embodiment of the present disclosure, various embodiments described above may be implemented as software including instructions stored in a computer-readable recording medium that may be read by a computer or a similar device by using software, hardware, or a combination thereof. In some cases, the embodiments described herein may be implemented by a processor itself. According to the software implementation, embodiments such as procedures and functions described herein may be implemented as separate software modules. Each software module may perform one or more functions and operations described herein.

A device-readable recording medium may be provided in the form of a non-transitory computer-readable recording medium. In this case, the term “non-transitory” only means that a storage medium is a tangible device and does not include a signal, but does not distinguish that data is stored semi-permanently or temporarily in the storage medium. In this regard, the non-transitory computer-readable medium refers to a medium that semi-permanently stores data, rather than a medium that stores data for a short moment, such as a register, a cache, a memory, etc., and may be read by a device. Specific examples of the non-transitory computer-readable medium may include a compact disc (CD), a digital versatile disc (DVD), a hard disk, a Blu-ray disk, a universal serial bus (USB), a memory card, a ROM, and the like.

As described above, the present disclosure has been described with reference to the embodiments illustrated in the drawings, which are merely for illustrative purposes, and those of ordinary skill in the art will understand that various modifications and other equivalent embodiments may be made therefrom. Therefore, the scope of the protection of the technology of the present disclosure should be determined by the technical spirit of the appended claims.

1. A deep-learning based diagnostic method of diagnosing an ischemic lesion of a coronary artery, the deep-learning based diagnostic method comprising:
obtaining an intravascular ultrasound (IVUS) image of a coronary artery lesion of a patient;
obtaining a mask image in which a vascular lumen is separated, by inputting the IVUS image into a first artificial intelligence model, and extracting an IVUS feature from the mask image; and
obtaining an FFR prediction value by inputting information including the IVUS feature into a second artificial intelligence model, and determining presence of an ischemic lesion.

2. The deep-learning based diagnostic method of claim 1, wherein the mask image is obtained by fusing pixels corresponding to an adventitia, a lumen, and a plaque of the coronary artery.

3. The deep-learning based diagnostic method of claim 1, wherein the IVUS feature includes a first feature and a second feature, and
the extracting of the IVUS feature further comprises extracting the first feature based on the mask image, and calculating and obtaining the second feature based on the first feature.

4. The deep-learning based diagnostic method of claim 1, wherein the information including the IVUS feature includes a clinical feature, and
the determining the presence of the ischemic lesion further comprises obtaining the FFR prediction value by inputting the IVUS feature and the clinical feature into the second artificial intelligence model, and determining the presence of the ischemic lesion.

5. The deep-learning based diagnostic method of claim 1, wherein the ischemic lesion determination operation further comprises, when the FFR prediction value of a coronary artery lesion is less than or equal to 0.80, determining the coronary artery lesion as an ischemic lesion.

6. A non-transitory computer-readable recording medium on which a program executable by a processor is recorded, to cause the processor to:
obtain an intravascular ultrasound (IVUS) image of a coronary artery lesion of a patient;
obtain a mask image in which a vascular lumen is separated, by inputting the IVUS image into a first artificial intelligence model, and extract an IVUS feature from the mask image; and
obtain an FFR prediction value by inputting information including the IVUS feature into a second artificial intelligence model, and determine presence of an ischemic lesion.

7. A diagnostic device for diagnosing an ischemic lesion of a coronary artery, the diagnostic device comprising a processor configured to:
obtain an intravascular ultrasound (IVUS) image of a coronary artery lesion of a patient;
obtain a mask image in which a vascular lumen is separated, by inputting the IVUS image into a first artificial intelligence model, and extract an IVUS feature from the mask image; and
obtain an FFR prediction value by inputting information including the IVUS feature into a second artificial intelligence model, and determine presence of an ischemic lesion.
